(19) United States
(12) Patent Application Publication
(10) Pub. No.: US 2026/0240789 A1
(43) Pub. Date: Aug. 20, 2026

(54) NOVEL DRUG DELIVERY SYSTEMS AND METHODS OF MAKING AND USING SAME

(71) Applicant: The Texas A&M University System, College Station, TX (US)

(72) Inventors: Srinath Palakurthi, Corpus Christi, TX (US); Rishi Paliwal, Kingsville, TX (US)

(73) Assignee: The Texas A&M University System, College Station, TX (US)

(21) Appl. No.: 19/162,552

(22) PCT Filed: Apr. 10, 2023
(86) PCT No.: PCT/US2023/018069

§ 371 Date: Sep. 5, 2025

(51) Int. Cl. A61K 9/51; A61K 9/19; A61K 38/13; A61K 45/06

(52) U.S. Cl. CPC A61K 9/5192; A61K 9/19; A61K 9/5138; A61K 9/5161; A61K 9/5169; A61K 38/13; A61K 45/06

(57) ABSTRACT

A method of preparing a nanoparticle-active pharmaceutical ingredient adduct comprising contacting a nanoparticle with a solvent under conditions suitable for the formation of a solubilized nanoparticle; contacting the solvent-nanoparticle mixture with an active pharmaceutical ingredient under conditions suitable for forming a solubilized nanoparticle-active pharmaceutical ingredient adduct; contacting the solubilized nanoparticle-active pharmaceutical ingredient adduct with an adduct stabilizing agent to form a stabilized nanoparticle-active pharmaceutical ingredient adduct; and; recovering the stabilized nanoparticle-active pharmaceutical ingredient adduct.

### CROSS-REFERENCE TO RELATED APPLICATIONS

This application is a 35 U.S.C. § 371 national stage application of PCT/US2023/018069 filed Apr. 10, 2023, and entitled “Novel Drug Delivery Systems and Methods of Making and Using Same,” which is hereby incorporated herein by reference in its entirety for all purposes.

### STATEMENT REGARDING FEDERALLY SPONSORED RESEARCH OR DEVELOPMENT

Not Applicable.

### TECHNICAL FIELD

The present disclosure generally relates to drug delivery mechanisms. More particularly, the present disclosure relates to novel nanoparticle systems for drug delivery.

### BACKGROUND

An active pharmaceutical ingredient (API) or drug is a substance intended for use in the diagnosis, cure, mitigation, treatment, or prevention of disease as per the Food and Drug Administration, FDA. Drug delivery is a technique of delivering medication such as an API to a patient in such a manner that specifically increases the drug concentration in some parts of the body as compared to others. The ultimate goal of any delivery system is to extend, confine, and target the drug in the diseased tissue with a protected interaction. Generally, drug delivery systems (DDS) overcome challenges associated with direct clinical use of the APIs such as the difficulty with the handling and accurate dosing of these materials. Further, the mode of administration of an API can affect the extent of patient compliance and adherence. Additionally, the extent to which APIs deteriorate after manufacturing due to factors such as light, moisture, temperature and pH make the use of delivery systems that stabilize or protect APIs attractive commercially.

### SUMMARY

Disclosed herein is a method comprising contacting a nanoparticle with a solvent under conditions suitable for the formation of a solvent-nanoparticle mixture; contacting the nanoparticle within the solvent-nanoparticle mixture with an active pharmaceutical ingredient under conditions suitable for forming a nanoparticle-active pharmaceutical ingredient adduct; contacting the nanoparticle-active pharmaceutical ingredient adduct with an adduct stabilizing agent to form a stabilized nanoparticle-active pharmaceutical ingredient adduct; and; recovering the stabilized nanoparticle-active pharmaceutical ingredient adduct.

Also disclosed herein is a method of preparing an immunosuppressive agent delivery system comprising contacting a zein nanoparticle with an alcohol under conditions suitable for the formation of a zein nanoparticle-alcohol mixture; contacting the zein nanoparticle within the zein nanoparticle-alcohol mixture with an immunosuppressive agent under conditions suitable for forming a zein nanoparticle immunosuppressive agent adduct wherein the immunosuppressive agent comprises cyclosporine A; contacting the zein nanoparticle immunosuppressive agent adduct with an adduct stabilizing agent to form a stabilized zein nanoparticle immunosuppressive agent adduct; and; recovering the stabilized zein nanoparticle immunosuppressive agent adduct.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIGS. 1A-1D depict results from the investigation of the morphology of cyclosporine A loaded zein nanoparticles (NP). A. Transmission Electron Micrograph (TEM; JEOL JEM-2010 TEM) of the freeze-dried NP (without cryoprotectant) negative staining with 1% phosphotungstic acid (PTA) at ×60,000 (FIG. 1A) and ×100,000 (FIG. 1B) magnification; B. TEM micrographs of the freeze-dried NP (with mannitol as cryoprotectant) at ×30,000 (FIG. 1C) and ×60,000 (FIG. 1D) magnification.

FIGS. 2A-2C depict overlays of Fourier Transform InfraRed (FTIR) spectra of (a) cyclosporine A (FIG. 2A), (b) Zein NP-loaded with cyclosporine A (FIG. 2B), (c) zein (FIG. 2C).

FIGS. 3A-3C depict overlays of differential scanning calorimetry (DSC) thermograms of zein (FIG. 3A), cyclosporine A (FIG. 3B), and Zein-cyclosporine A (FIG. 3C).

FIG. 4 depicts a graph of the pH-dependent in-vitro drug release profile of cyclosporine A loaded zein nanoparticles in simulated gastric fluid (SGF, pH 1.5), simulated intestinal fluid (SIF, pH6.5) and phosphate buffered saline (PBS, pH 7.4), by dialysis method.

FIGS. 5A-5D depict analyses of the efficacy of NANOCYCLO formulation in DSS-induced colitis mice based on the following parameters: body weight (FIG. 5A) monitored on daily basis, and on day 8 the mice were euthanized on day 8, and then colon length was measured (FIG. 5B), the colon:body weight were measured (FIG. 5C); and the liver:body weight was measured (FIG. 5D).

FIG. 6 depicts a comparison of pro-inflammatory and anti-inflammatory cytokine levels in the colitis mice treated with NANOCYCLO and NEORAL formulations with the graphs shown for IL-β (FIG. 6A); IL-α (FIG. 6B); IL-10 (FIG. 6C); G-CSF (FIG. 6D); IL-17A (FIG. 6E); and TNFα (FIG. 6F). Results are expressed as mean±SD (n=5) and the data were analyzed by one-way ANOVA. p<0.05 was considered significant.

### DETAILED DESCRIPTION

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Groups of elements of the periodic table are indicated using the numbering scheme indicated in the version of the periodic table of elements published in Chemical and Engineering News, 63(5), 27, 1985. In some instances, a group of elements can be indicated using a common name assigned to the group; for example alkali earth metals (or alkali metals) for Group 1 elements, alkaline earth metals (or alkaline metals) for Group 2 elements, transition metals for Group 3-12 elements, and halogens for Group 17 elements.

Regarding claim transitional terms or phrases, the transitional term “comprising”, which is synonymous with “including,” “containing,” “having,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the subject matter described herein. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter the composition or method to which the term is applied. When a claim includes different features and/or feature classes (for example, a method step, feedstock features, and/or product features, among other possibilities), the transitional terms “comprising,” “consisting essentially of,” and “consisting of” apply only to the feature class which is utilized and it is possible to have different transitional terms or phrases utilized with different features within a claim. For example, a method can comprise several recited steps (and other non-recited steps).

Within this specification, use of “comprising” or an equivalent expression contemplates the use of the phrase “consisting essentially of,” “consists essentially of,” or equivalent expressions as alternative aspects to the open-ended expression. Additionally, use of “comprising” or an equivalent expression or use of “consisting essentially of” in the specification contemplates the use of the phrase “consisting of,” “consists of,” or equivalent expressions as an alternative to the open-ended expression or middle ground expression, respectively. For example, “comprising” should be understood to include “consisting essentially of,” and “consisting of” as alternative aspects for the aspect, features, and/or elements presented in the specification unless specifically indicated otherwise.

The terms “a,” “an,” and “the” are intended, unless specifically indicated otherwise, to include plural alternatives, e.g., at least one. For any particular compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise; e.g., a general reference to pentane includes n-pentane, 2-methyl-butane, and 2,2-dimethylpropane while a general reference to a butyl group includes an n-butyl group, a sec-butyl group, an iso-butyl group, and a tert-butyl group. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

Features within this disclosure that are provided as minimum values can be alternatively stated as “at least” or “greater than or equal to” any recited minimum value for the feature disclosed herein. Features within this disclosure that are provided as maximum values can be alternatively stated as “less than or equal to” for the feature disclosed herein.

Use of the term “optionally” with respect to any element of a claim is intended to mean that the subject element is required, or alternatively, is not required. Both alternatives are intended to be within the scope of the claim

Disclosed herein are methods and compositions for use as drug delivery systems (DDS). In one or more aspects, the DDS comprises a material derived from a renewable resource such as from a plant. A DDS of the present disclosure may further be characterized as comprising nanoparticles of any suitable shape. Herein a nanoparticle refers to a particle of matter having at least one dimension between about 1 nm to about 350 nm or, alternatively from about 500 nm to about 800 nm.

In an aspect, a DDS comprises a protein, alternatively a plant protein. The DDS may be amphiphilic comprising both hydrophobic and hydrophilic regions. In one or more aspects, the DDS is largely hydrophobic having a ratio of hydrophobic:hydrophilic regions of about 75:25, alternatively about greater than about 50:less than about 50. A DDS of the type disclosed herein may be characterized by a particle size ranging from about 120 nm to about 800 nm, alternatively from about 200 nm to about 700 nm or, alternatively from about 250 nm to about 600 nm, a polydispersity index (PDI) of from about 0.005 to about 0.4, alternatively from about 0.01 to about 0.4, alternatively from about 0.005 to about 0.212 or, alternatively from about 0.1 to about 0.4; and a zeta potential of from about −15 millivolts (mv) to about 30 mv, alternatively from about −10 mv to about 30 mv or, about −5 mv to about 30 mv. As will be understood by one of ordinary skill in the art, the exact zeta potential will be dependent to some extent on the specific API.

In an aspect, the DDS comprises zein. Zein refers to a class of prolamine protein found in maize (corn) where it is the major protein comprising from about 35% to about 60% of the total protein in corn and is a generally regarded as safe (GRAS status) food additive by the FDA. Zein is usually manufactured as a powder from corn gluten meal. Pure zein is clear, odorless, tasteless, hard, water-insoluble, and edible, and it has a variety of industrial and food uses. Zein has a unique amino acid profile consisting of three parts of hydrophobic amino acids with one part of hydrophilic amino acid residues. Zein can form a ribbon like structure with hydrophobic domains at the front and back regions, with hydrophilic edges. These structural features allow zein to function as a polymeric amphiphile with a brick-like structure. Zein has a solubility in alcohol solutions ranging from about 60% to about 90% volume by volume (v/v).

Hereinafter the disclosure will refer to the use of a nanoparticle prepared using zein, however the present disclosure contemplates the use of other nanoparticles derived from renewable resources which may be processed using any suitable methodology to obtain nanoparticles that can function with a DDS of the type disclosed herein.

In an aspect, a DDS of the type disclosed herein is prepared for use as an oral DDS. A method of preparing an oral DDS of the type disclosed herein comprises introducing a zein particle to a suitable solvent to form a solvent nanoparticle mixture (SNM) or more particularly a zein solvent-nanoparticle mixture (SZNM), for example an alcohol. The method may further comprise liquid-liquid dispersion by contacting an API of interest with the nanoparticle, in solution, with agitation to form a reaction media. Contacting of the nanoparticle and API in the reaction media may occur for a time period suitable to allow the zein nanoparticle to associate with the API and form generally a nanoparticle active pharmaceutical ingredient adduct and more particularly a zein nanoparticle active pharmaceutical ingredient adduct designated, ZNP-API. As will be understood by one of ordinary skill in the art, the ratio of ZNP to API used can be determined by one of ordinary skill in the art depending on factors such as the nature of the API, the solution and the nature of the nanoparticle. For example, in the case of a ZNP-API the ratio of ZNP to API may be about 1 to about 0.3, or alternatively about 1 to about 0.15.

The resulting ZNP-API adduct may be treated with an adduct stabilizing agent capable of facilitating increased interaction between the ZNP and API to form a stabilized nanoparticle active pharmaceutical ingredient adduct. Herein the adduct stabilizing agent stabilizes the newly formed ZNP-API adduct, but may also play a role in the development of additional formulations and may affect drug bioavailability.

In one or more aspects, the adduct stabilizing agent comprises polymers, surface active agents or combinations thereof. Nonlimiting examples of compounds that may be used as an adduct stabilizing agent include sodium lauryl sulfate, polyvinyl pyrrolidone, PLURONICS F68, PLURONICS F127, TWEEN 80, hydroxypropyl methylcellulose, polyvinyl alcohol, polyethylene glycol, cyclodextrin, tocopherol, succinate, hydroxyethyl cellulose, methylcellulose, carboxymethylcellulose sodium, sodium alginate and combinations thereof. An amount of adduct stabilizing agent suitable for use in the present disclosure may range from about 1 weight percent (wt. %) to about 4 wt. % based on the total weight of the adduct, alternatively about 1 wt. % to about 2 wt. % or alternatively about 2 wt. % to about 4 wt. %. In an aspect, an adduct stabilizing agent suitable for use in the present disclosure comprises polyvinyl pyrrolidone and cyclodextrin; where the polyvinyl pyrrolidone to cyclodextrin ratio is about 1:4, alternatively about 1:3 or, alternatively about 1:2. The stabilized ZNP-API may be reacted until sufficient solvent evaporation occurs to allow for precipitation of the stabilized ZNP-API.

A stabilized ZNP-API may be isolated from the reaction media and unreacted API using any suitable solid-liquid separation methodology. For example, the precipitated ZNP-API may be isolated by centrifugation.

In one or more aspects, the precipitated isolated ZNP-API may be treated with a cryopreservation agent. Nonlimiting examples of cryopreservation agents suitable for use in the present disclosure include dimethyl sulfoxide, mannitol, ethylene glycol, glycerol, 2-methyl-2,4-pentanediol, propylene glycol, sucrose, trehalose or combinations thereof. In one or more aspects, the precipitated isolated ZNP-API is treated with the cryopreservation agent mannitol to produce a cryo ZNP-API.

In one or more aspects, the cryo ZNP-API is lyophilized to produce a lyophilized ZNP-API, designated lyo-ZNP-API. Lyophilization and freeze drying are synonymous and refer to a water removal process typically used to preserve perishable materials, to extend shelf life or, make the material more convenient for transport. Lyophilization works by freezing the material, then reducing the pressure and adding heat to allow the frozen water in the material to sublimate.

In one or more aspects, the lyo-ZNP-API is further processed to meet one or more user and/or process objectives such as through the inclusion of additives conventionally found in pharmaceutical formulations. In an aspect, the composition and/or formulation may be combined into a formulation with additional ingredients (additives). As used herein, additives include, but are not limited to, one or more of the following: excipients; dispersing agents; inert diluents; granulating and disintegrating agents; binding agents; lubricating agents; coloring agents; preservatives; physiologically-degradable compositions such as gelatin; suspending agents; dispersing or wetting agents; emulsifying agents, demulcents; buffers; salts; thickening agents; fillers; and antifungal agents.

In one or more aspects, any suitable API may be used to prepare a lyo-ZNP-API of the type disclosed herein. Nonlimiting examples of APIs that may be used to prepare a lyo-ZNP-API include antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, receptor blockers and reuptake modulators, beta-adrenergic blockers, calcium channel blockers, muscle relaxants, analgesics, antipyretics, stimulants, anticholinesterase agents, parasympathomimetic agents, hormones, anticoagulants, antithrombotics, thrombolytics, immunosuppressants, hormone agonists/antagonists, vitamins, antineoplastics, cathartics, diuretics, fungicides, ectoparasiticides, antiparasitics, alkaloids, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, anti-inflammatories, wound care products, antithrombogenic agents, antiangiogenic agents, antigenic agents, rejection/anti-rejection drugs, antifungal agents, antiviral agents, antibiotics, cholesterol-reducing drugs, antitussives, histamine-blocking drugs, monoamine oxidase inhibitor, or combinations thereof.

Nonlimiting examples of APIs that may be suitable for use in the formation of a I-ZNP-API include silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, Cyclosporin, antimicrobial peptides (e.g., natural and synthetic origin), NEOSPORIN® (i.e., Bacitracin, Polymyxin B, and Neomycin), POLYSPORIN® (i.e., Bacitracin and Polymyxin B), Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate, Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane, Dipyrone; Doxpicomine Hydrochloride: Drinidene; Enadoline Hydrochloride; Epirizole; Ergotamine Tartrate; Ethoxazene Hydrochloride; Etofenamate; Eugenol; Fenoprofen; Fenoprofen Calcium: Fentanyl Citrate; Floctafenine; Flufenisal; Flunixin; Flunixin Meglumine; Flupirtine Maleate, Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Loinoxicam; Magnesium Salicylate; Mefenarnic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride, Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride, Salcolex, Salethamide Maleate; Salicylamide; Salicylate Meglumine; Salsalate; Sodium Salicylate; Spiradoline Mesylate; Sufentanil; Sufentanil Citrate; Talmetacin; Talniflumate, Talosalate; Tazadolene Succinate; Tebufelone; Tetrydamine; Tifurac Sodium, Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin, Aflyzosin Hydrochloride: Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium, Cicletanine; Cilazapril; Clonidine; Clonidine Hydrochloride; Clopamide; Cyclopenthiazide; Cyclothiazide; Darodipine, Debrisoquin Sulfate; Delapril Hydrochloride; Diapamide; Diazoxide; Dilevalol Hydrochloride; Diltiazem Malate; Ditekiren; Doxazosin Mesylate, Eeadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride, Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride, Pazoxide; Pelanserin Hydrochloride; Perindopril Erbumine; Phenoxybenzamine Hydrochloride; Pinacidil; Pivopril; Polythiazide; Prazosin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride: Quinuclium Bromide; Ramipril; Rauwolfia Serpentina; Reserpine, Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Tierynafen; Tinabinol; Tiodazosin; Tipentosin Hydrochloride; Trichlormethiazide; Trimazosin Hydrochloride; Trimethaphan Camsylate; Trimoxamine Hydrochloride, Tripamide; Xipamide; Zankiren Hydrochloride; Zofenoprilat Arginine, Alclofenac; Alclometasone Dipropionate; Algestone Acetonide; Alpha Amylase; Ameinafal; Ameinafide; Amfenac Sodium: Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium: Diflunisal; Difluprednate; Diftalone, Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; FlufenamicAcid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; lbuprofen; Ibuprofen Aluminum; Ibuprofen Piconol; Ilonidap: Indomethacin; Indomethacin Sodium; Indoprofen; Indoxole: Intrazole: lsoflupredone Acetate; lsoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone, Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex, Salnacedin; Salsalate; Sanguinarium Chloride: Seclazone; Sermetacin; Sudoxicam; Sulindac; Suprofen; Talmetacin; Talniflumate; Talosalate; Tebufelone; Tenidap; Tenidap Sodium, Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium or combinations thereof.

In one or more aspects, a lyo-ZNP-API of the present disclosure comprises the immunosuppressive agent cyclosporine as an API. In such aspects, the lyo-ZNP-API may useful in the treatment of inflammatory bowel disease (IBD). IBD is an idiopathic inflammatory disease commonly prevalent in the form of Crohn's disease (CD) or ulcerative colitis (UC), and is believed to result from an abnormal response of intestinal immune cells when exposed to bacterial antigens via a compromised luminal barrier. IBD is characterized by episodes of abdominal pain, diarrhea, bloody stools, weight loss, inflammation with ulceration and a variable degree of mucosal damage. Cyclosporine A (CsA) is an immunosuppressant drug that has been used as a rescue therapy in acute flare up and in steroid-refractory UC owing to its rapid onset of action. Acute remission rates with CsA are found to be in the range of 63 to 82%. However, its long term usage is known to induce several potential side effects, including nephrotoxicity, hypertension, seizures and neurotoxicity. Systemic CsA therapy in clinical practice needs careful monitoring of drug plasma concentration to prevent the associated side effects such as nephrotoxicity, hypertension, seizures, paresthesias, hyperkalemia and gingival swelling. Moreover, the currently used CsA formulations contain polyoxyethylated castor oil, CREMOPHOR EL®, which may cause nephrotoxicity and anaphylactic reactions. Rectal enemas and intrathecal delivery routes were also investigated as the alternative to the oral/intravenous administration, to reduce the systemic toxicity. However rectal/intrathecal delivery of drugs is suitable for only treating the distal region of the colon and it has poor patient acceptance. Consequently, disclosed herein is a lyo-ZNP-CsA novel formulation that targets the mucosal inflammatory sites minimizing systemic drug exposure.

### ADDITIONAL DISCLOSURE

A first aspect which is a method of preparing a nanoparticle-active pharmaceutical ingredient adduct comprising contacting a nanoparticle with a solvent under conditions suitable for the formation of a solvent-nanoparticle mixture; contacting the nanoparticle within the solvent-nanoparticle mixture with an active pharmaceutical ingredient under conditions suitable for forming a nanoparticle-active pharmaceutical ingredient adduct; contacting the nanoparticle-active pharmaceutical ingredient adduct with an adduct stabilizing agent to form a stabilized nanoparticle-active pharmaceutical ingredient adduct; and; recovering the stabilized nanoparticle-active pharmaceutical ingredient adduct.

A second aspect which is the method of the first aspect wherein the active pharmaceutical ingredient comprises antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, receptor blockers and reuptake modulators, beta-adrenergic blockers, calcium channel blockers, muscle relaxants, analgesics, antipyretics, stimulants, anticholinesterase agents, parasympathomimetic agents, hormones, anticoagulants, antithrombotics, thrombolytics, immunosuppressants, hormone agonists/antagonists, vitamins, antineoplastics, cathartics, diuretics, fungicides, ectoparasiticides, antiparasitics, alkaloids, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, anti-inflammatories, wound care products, antithrombogenic agents, antiangiogenic agents, antigenic agents, rejection/anti-rejection drugs, antifungal agents, antiviral agents, antibiotics, cholesterol-reducing drugs, antitussives, histamine-blocking drugs, monoamine oxidase inhibitors, or combinations thereof.

A third aspect which is the method of any of the first through second aspects wherein the active pharmaceutical ingredient comprises an immunosuppressive agent.

A fourth aspect which is the method of any of the first through third aspects wherein the nanoparticle has a particle size ranging from about 120 nm to about 800 nm.

A fifth aspect which is the method of any of the first through fourth aspects wherein the nanoparticle has a polydispersity index of from about 0.005 to about 0.4.

A sixth aspect which is the method of any of the first through fifth aspects wherein the nanoparticle has a zeta potential of from about −15 mv to about +30 mv.

A seventh aspect which is the method of any of the first through sixth aspects wherein contacting occurs with continuous agitation.

An eighth aspect which is the method of any of the first through seventh aspects, wherein recovering the stabilized nanoparticle-active pharmaceutical ingredient adduct comprises a solid-liquid separation method.

A ninth aspect which is a method of any of the first through eighth aspects wherein the adduct stabilizing agent comprises polymers, surface active agents or combinations thereof.

A tenth aspect which is the method of any of the first through ninth aspects wherein the adduct stabilizing agent comprises sodium lauryl sulfate, polyvinyl pyrrolidone, PLURONICS F68, PLURONICS F127, TWEEN 80, hydroxypropyl methylcellulose, polyvinyl alcohol, polyethylene glycol, cyclodextrin, tocopherol, succinate, hydroxyethyl cellulose, methylcellulose, carboxymethylcellulose sodium, sodium alginate or combinations thereof.

An eleventh aspect which is the method of any of the first through tenth aspects further comprising lyophilizing the stabilized nanoparticle-active pharmaceutical ingredient adduct in the presence of a cryopreservation agent.

A twelfth aspect which is the method of the eleventh aspect wherein the cryopreservation agent comprises dimethyl sulfoxide, mannitol, ethylene glycol, glycerol, 2-methyl-2,4-pentanediol, propylene glycol, sucrose, trehalose or, combinations thereof.

A thirteenth aspect which is a method of preparing a drug delivery system comprising contacting a zein nanoparticle with an alcohol under conditions suitable for the formation of a solvent-zein nanoparticle mixture; contacting zein nanoparticle within the solvent-zein nanoparticle mixture with an immunosuppressive agent under conditions suitable for forming a zein nanoparticle immunosuppressive agent adduct wherein the immunosuppressive agent comprises cyclosporine A; contacting the zein nanoparticle immunosuppressive agent adduct with an adduct stabilizing agent to form a stabilized zein nanoparticle immunosuppressive agent adduct; and; recovering the stabilized zein nanoparticle immunosuppressive agent adduct.

A fourteenth aspect which is the method of the thirteenth aspect wherein the solvent comprises methanol, ethanol or combinations thereof.

A fifteenth aspect which is the method of any of the thirteenth through fourteenth aspects wherein the polydispersity index of the zein nanoparticle ranges from about 0.005 to about 0.212.

A sixteenth aspect which is the method of any of the thirteenth through fifteenth aspects wherein the adduct stabilizing agent comprises a mixture of polyvinyl pyrrolidone and cyclodextrin.

A seventeenth aspect which is the method of any of the thirteenth through sixteenth aspects further comprising lyophilizing the stabilized zein nanoparticle immunosuppressive agent adduct in the presence of a cryopreservation agent.

An eighteenth aspect which is the method of any of the thirteenth through seventeenth aspects wherein the cryopreservation agent comprises mannitol,

A nineteenth aspect which is the method of any of the thirteenth through eighteenth aspects wherein the ratio of zein nanoparticle to cyclosporine A is about 1:0.3

A twentieth aspect which is a method of treating a subject comprising administering a formulation comprising the stabilized zein nanoparticle immunosuppressive agent adduct of claim any of the thirteenth through ninteenth aspects.

### EXAMPLES

The following examples are provided to illustrate the present disclosure. The examples are not intended to limit the scope of the present disclosure and they should not be so interpreted.

### Materials and Methods

### Materials

Zein F-4000 (yellow zein) was purchased from Flo Chemical Corporation, Ashburnham, MA, USA. Cyclosporine A was obtained from Euroasia's Inc., Mumbai, India, and was used as received. Polyvinylpyrrolidone (average molecular weight 40,000 Da), D-Mannitol and ethanol were from Sigma-Aldrich (St. Louis, MO, USA). Tween-20 was from Acros (NJ, USA) and HPLC grade solvents (methanol and acetonitrile) were obtained from Fisher-Scientific (Houston, TX, USA). Dialysis membranes were purchased from Spectrum Laboratories (Rancho Dominguez, CA, USA). Citrucel was purchased from GSK Consumer Healthcare (Warren, NJ, USA). Radioimmunoprecipitation assay buffer (RIPA buffer) and Bicinchoninic acid (BCA) assay kit were purchased from Fisher Scientific (Waltham, MA, USA). Multi-Analyte ELISArray Kit was obtained from Qiagen (German town, MD, USA).

### Preparation of CsA Loaded Zein Nanoparticles

Zein particles were prepared using a modified liquid-liquid dispersion method optimized in our laboratory. Precisely, zein (25 mg) was dissolved in 10 mL of ethanol/water binary solvent (75:25 v/v) to form a stock solution, and CsA solution in methanol (10 mg/mL) was added to zein solution. A mixture of polyvinylpyrrolidone (PVP-40; 4% w/v) and methyl β-cyclodextrin (1% w/v) solution in water (5 mL) was added into zein-CsA solution under continuous stirring using a magnetic stirrer, followed by the addition of 10 mL deionized water. The dispersion formed was stirred overnight to allow the evaporation of ethanol. The dispersions were then subjected to centrifugation at 8500 rpm for 45 min and the supernatant was removed to collect the nanoparticle (NP). NP were dispersed in 10 mL of mannitol solution in water (5% w/v) and then freeze-dried for 24 h to obtain solid NPs powder samples.

### Particle Size Analysis

Hydrodynamic diameter, polydispersity index (PDI) and zeta potential of NP was measured by dynamic light scattering (DLS) using ZetaPALS zeta potential analyzer (Brookhaven Instruments Corp., Holtsville, NY, USA). Particle size analysis and zeta potential were performed at 25° C., after dispersing samples in deionized water.

### Transmission Electron Microscopy (TEM)

The shape and surface morphology of the zein and zein-CsA NP was determined by Transmission Electron Microscopy (JEOL JEM-2010 TEM). NP suspension and freeze dried NP samples was redispersed in deionized water and dried on a carbon-coated copper grid before TEM analysis.

### Fourier Transform Infrared (FTIR) Spectroscopy

Infra-red spectra of CsA and the freeze dried samples were recorded using potassium bromide disk method. Samples were mixed with dry powdered potassium bromide and compressed into transparent disk under high pressure and spectrum was recorded in attenuated total reflectance (ATR) mode in the wave number region 4,000-600 cm⁻¹ using Shimadzu-IR Prestige 21 Fourier Transform Infrared spectrophotometer.

### Differential Scanning Calorimetry Studies

Differential Scanning Calorimetry (DSC) analysis was performed using a TI Q200 DSC apparatus (TA instruments, New Castle, DE, USA). Two milligrams of the sample was sealed in aluminum pans and scanned from 20° C. to 300° C. at a ramp rate of 10° C./min under nitrogen purge at a flow rate of 50 mL/min. An empty aluminum pan was used as the reference.

### HPLC Analysis of CsA

Concentration of CsA in the analytical samples was determined using a Shimadzu HPLC system (Milford, USA), equipped with a LC-20AB solvent pump, SIL-20A HT autosampler, CTO-20A column temperature oven and a modelSPD-20A UV/vis detector. Separations were accomplished on a Phenomenex C18 column (250×4.6 mm id, 5 μm particles) at 60° C. Mobile phase consisted of acetonitrile (ACN) and water (75:25%) at a flow rate of 1.0 mL min⁻¹. The injection volume was 20 μL, the absorbance was measured at a wavelength of 210 nm and the total chromatographic run time was 12.0 min. On-line quantitation of the HPLC results was obtained using Shimadzu Lab solution software. Linearity of the HPLC-UV method for the determination of CsA was evaluated by a calibration curve in the concentration range of 0.25-2.5 μg/mL. The calibration curve was obtained by plotting the peak-area ratio versus the analyte concentration prepared.

### Encapsulation Efficiency (EE)

Encapsulation efficiency is the percentage of initial drug amount that is encapsulated into the NP. For calculating EE, 1 mg of lyophilized NP samples containing CsA was dispersed in 10 mL of acetonitrile (ACN) and shaken on an orbital shaker (350 rpm) for 12 h at room temperature. Thereafter ACN solution with dissolved CsA was carefully removed and centrifuged at 8000 rpm for 15 min to remove any protein precipitate. Supernatant so obtained was filtered through nylon syringe filters (0.4 μM) and diluted appropriately with ACN before making CsA quantification through a pre-established HPLC method. Encapsulation efficiency was determined using the following equation:

$$EE ( \% ) = \frac{Total CsA amount - Free CsA amount}{Total CsA amount} \times 100$$

Loading capacity (LC) is the amount of the drug loaded per unit weight of the NP. LC may be calculated using the following equation:

$$LC ( \% ) = Encapsulated CsA weight \times 100 Nanoparticles weight$$

### In Vitro Release Experiments

Cyclosporine A release from zein NP was tested in simulated gastric fluid (SGF) and simulated intestinal fluid (SIF) by dialysis method. SGF containing 0.2% NaCl, 0.063 M HCl, 3.2 mg/mL pepsin at pH 1.2 and SIF containing 0.2% NaCl, 3.2 mg/mL pancreatin in 20 mM potassium phosphate buffer pH6.8 were prepared as reported earlier, One milligram of freeze dried NP were dispersed in 45 mL of either stimulated gastric fluid (SGF; pH 1.2), stimulated intestinal fluid (SIF; pH 6.8) and phosphate buffered saline (PBS; pH 7.4) at 25° C. in a conical centrifuge tubes. An aliquot (100 μL) of the supernatant was periodically removed (0, 1, 2, 3, 4, 5, 6, 24 h) while the same volume of medium was replaced to keep the total volume constant. CsA concentration in the supernatant solution was determined by HPLC, Cumulative amount released vs time was plotted.

### DSS-Induced Colitis Model

Six- to 8-week-old C57BL/6 mice (WT) were purchased from The Jackson Laboratory and bred in-house to generate the experimental mice. Male mice weighing 20-25 g were used for the experiments. The animals were housed in a specific pathogen-free facility in individually ventilated cages. Acute colitis was induced. Briefly, mice with either NEORAL or NANOCYCLO were fed with 5% (w/v) DSS in drinking water over a period of 8 days or loss of ≥25% of initial body weight whichever came first. This procedure is known to induce colonic epithelial injury. This was followed by a recovery phase of 1 week where DSS was discontinued. The UC induced mice were divided into 4 groups of each six mice (n=6). CsA, Neoral® and CsA-loaded zein NP (NANOCYCLO) were administered as a dispersion in 3% w/v citrucel in water at a dose 2 mg/kg to three separate groups, starting on day 7. The fourth group served as the negative control (PBS pH 7.4). Animals were provided unlimited access to food and water throughout the experiment (ad libitum). Body weights of mice were measured daily. The mice were euthanized following recovery phase, and all the data shown are based on this end-point. Following treatment, the distal colon and serum were sampled for analysis. Images were taken by a NIKON camera. Quantitation of colon length, colon to body weight ratio, length of cecum, and crypt lengths were calculated using NIS element imaging software (NIKON®).

Evaluation of colitis severity: Clinical disease activity was monitored daily using a disease activity index (DAI) using three parameters; weight loss, stool consistency and anal bleeding, as described previously. An earlier validated clinical disease activity index was calculated according to the following parameters: stool consistency (0-4), the presence or absence of fecal blood (0-4), and body weight loss (0-4). The maximum possible score taken was 12. Survival reflects the time required for animals to reach an end point requiring euthanasia, including loss of mobility, weight loss exceeding 30%, severe rectal prolapse. Percentage of survival vs. time data will be analyzed using Kaplan-Meier analysis and compared using the long-rank test. Survival reflects the time required for animals to reach an end point requiring euthanasia, including loss of mobility, weight loss exceeding 30%, severe rectal prolapse. Percentage of survival vs. time data will be analyzed using Kaplan-Meier analysis and compared using the long-rank test.

Colon length, Colon-to-liver and colon-to-spleen ratios: All the mice were euthanized on the day 8 from the day of start of the treatment (one day after the last treatment). Blood and the entire colons from cecum to the anus was resected. Colon length and weight were measured. In addition to the colon, liver and spleen was separated, rinsed with water to remove the residual blood, blotted on filter paper and weighed. The weight ratios of colon to liver, and colon to spleen were calculated, and used as the indicators of inflammation.

Quantification of cytokines by enzyme-linked immunosorbent assay (ELISA): Concentration of proinflammatory cytokines (tumor necrosis factor; TNF-α, IL-1β, IL-1α, Interferon gamma; IFN-γ and IL-17A, Granulocyte macrophage colony stimulating factor; GM-CSF) anti-inflammatory cytokines (IL-10) in the colon samples was determined by a sandwich ELISA using Multi-Analyte ELISArray Kit (SA Biosciences) according to the manufacturer's instructions. The absorbance at 450 nm was measured using a microplate reader (Awareness Technology Inc.), Briefly, colon samples were cut into small pieces whilst keeping on ice and homogenized with radioimmunoprecipitation assay buffer (RIPA buffer) containing protease inhibitor. The samples were sonicated keeping on ice to break the tissue further and centrifuged at 2500×g at 4° C. for 5 min. The supernatants were collected and centrifuged at 10,000×g, 4° C. for 10 min and the collected supernatants were subjected to ELISA. Total protein concentration in the samples was determined by Bicinchoninic acid (BCA) assay kit.

Quantification of CsA in colon and serum samples: Blood samples collected into the heparinized tubes were centrifuged at 2000 rpm at 4° C. for 4 min and the serum will be collected and stored at −20° C. until further analysis. CsA concentration in the serum and colon tissue lysate samples was determined using an established HPLC method.

Statistical analysis: Statistical analysis of the in vitro cell line experiments and in vivo efficacy study was performed using ANOVA with general linear model (Graph Pad InStat software). P<0.05 was considered statistically significant.

### Results

### Nanoparticle Characterization

Zein was dissolved in 75% ethanol and the stock zein solution was sheared into bulk deionized water containing optimized amount of the stabilizers/surfactants. Due to the excellent miscibility of ethanol and water, ethanol in the dispersed droplets partitions into the bulk water-solvent attrition and insoluble zein precipitates to form nanoparticles. In this study CsA loaded zein NP were prepared by oil-in water emulsion/solvent evaporation method. Particle size is a important factor in the development of the NP formulations for the treatment of ulcerative colitis because it affects accumulation of particles in the inflammatory regions. Evidence suggests that NP exhibit size-dependent adhesion to the mucosa and the smaller particles (200 nm) penetrate the strong mucus layer surrounding the ulcerated tissues, and are rapidly taken by the macrophages in the inflamed regions. Further, ulcerated tissues contain high concentrations of positively charged proteins that increase the affinity of negatively charged particles.

In this study, several surfactants/stabilizers (including PVP-40, TWEEN-20, PLURONIC F-68) were used and the stability of NPs so obtained was evaluated. From our initial studies, PVP-40 at 4%, was found to be an effective stabilizer in preventing the aggregation of NP via the repulsive forces that arise from its hydrophobic carbon chains that extend into solvent. Table 1 shows the effect of concentration of PVP-40 on the average particle size of the CsA loaded zein NP.

TABLE 1

| | | PVP- | Size(nm)/PDI | | Size(nm)/PDI |
|---|---|---|---|---|---|
| | Zein | 40 | (before | Size(nm)/PDI | (after freeze |
| Sample | (in | (in | freeze | (after freeze | drying with |
| code | mg) | mg) | drying) | drying) | Mannitol) |
| Z0 | 25 | 0 | 192/0.052 | No | — |
| | | | | re-dispersion | |
| Z1 | 25 | 40 | 179/0.052 | No | — |
| | | | | re-dispersion | |
| Z2 | 25 | 100 | 176/0.281 | 853/0.209 | — |
| | | | | (unstable) | |
| Z3 | 25 | 200 | 223/0.102 | 654/0.160 | 425/0.317 |
| Z4 | 25 | 400 | 165/0.067 | 524/0.117 | 347/0.178 |

Particle size of the NP prepared with and without PVP-40 as the stabilizer was 165 and 192 nm, respectively. Moreover, NP suspension prepared without PVP-40 was not easily redispersible (aggregation of NP) when the suspension was subjected to centrifugation to collect the NP. To prevent NP aggregation, PVP-40 was added in increasing concentration and the NP formulation with average particle of 165 nm with polydispersity index ((PDI) of 0.067 was chosen for further investigation.

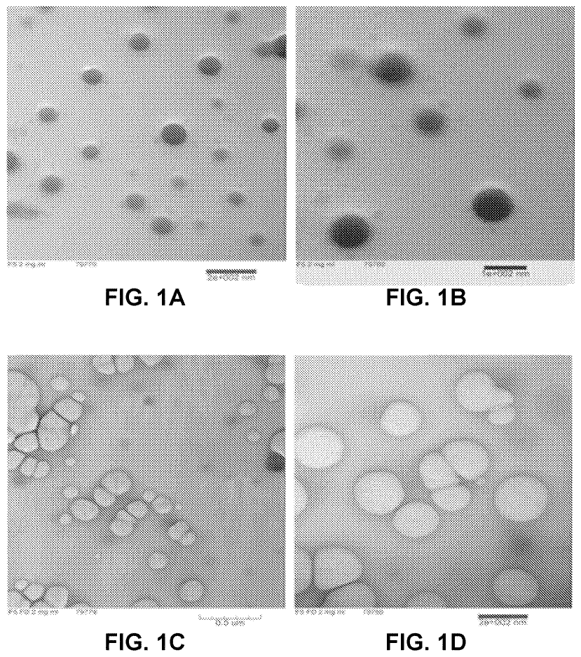

To render shelf stability to the NP, zein NP were subjected to free-drying process. Cyroprotectants are commonly used to facilitate re-dispersion of freeze dried NPs in an aqueous system. Specifically, mannitol was used as cryoprotectant and the mean particle size before and after freeze-drying process is summarized in Table 1. As evident from the results, freeze drying process affected the particle size and dispersibility of zein NPs. Particles prepared in absence of PVP could not be redispersed, while particle prepared with PVP-40 (Z3 and Z4) as stabilizer and mannitol as cryoprotectant, was redispersible. The TEM images of the zein NP before and after freeze drying process are presented in FIG. 1. Particles were mostly spherical with diameter less than 200 nm and smooth surface, however with significant change in the surface morphology (or texture) of the particles before and after freeze drying process. Zeta potential of zein NP was highly negative at pH 7 which changed to positive values when pH decreased to 2, with zero value at pH 6. These results may be accounted for the known isoelectric point (pI) of zein 6.2.

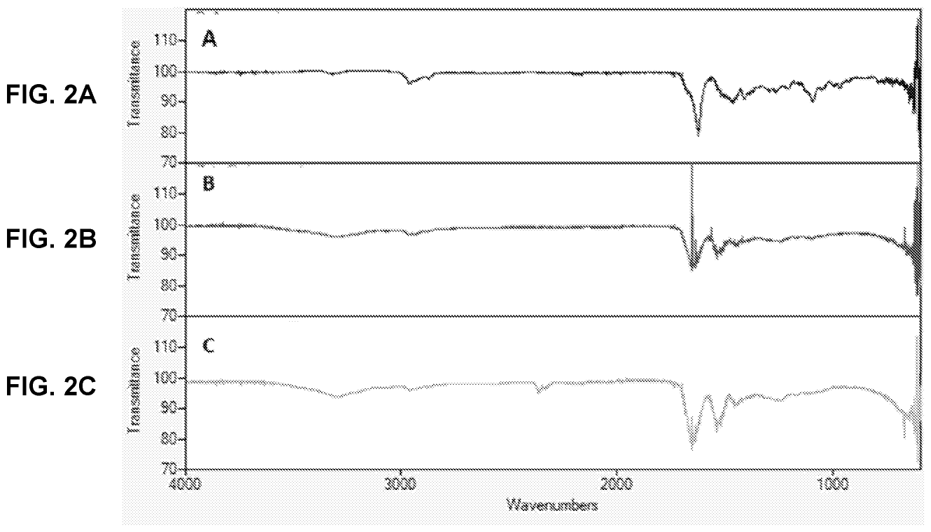

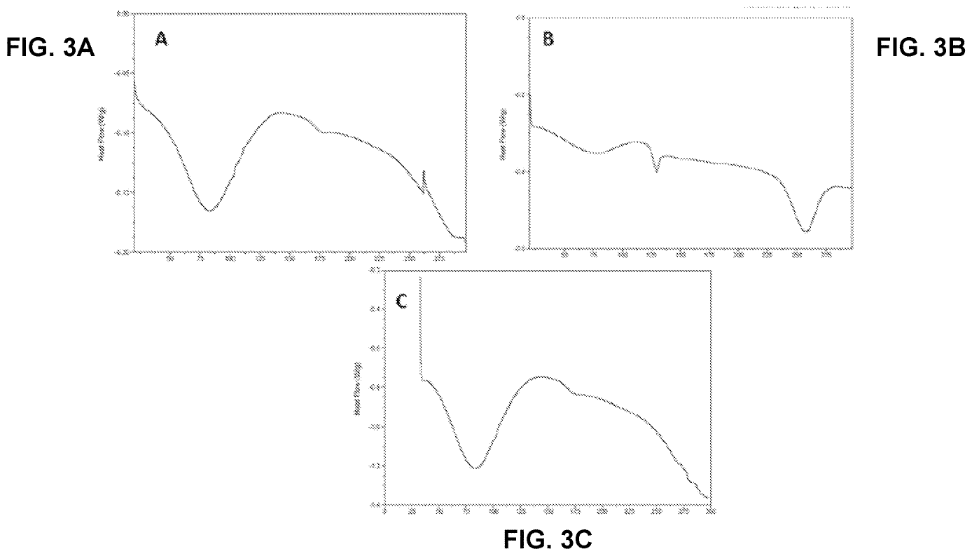

Because of the similarity in chemical properties, zein (hydrophobic protein) and CsA (hydrophobic peptide), potential protein-peptide interactions were tested using spectroscopic and thermal analysis. Fourier transform infrared (FTIR) spectrum of zein shows characteristic peaks around 1,640, 1,540, and 1,230 cm⁻¹ corresponding to amide 1, 11, and 111, respectively as shown in FIG. 2. The FTIR spectrum from zein-CsA represents a similar profile as compared to spectra obtained from zein NP, except additional peak at 1640 cm⁻¹, possibly suggesting an interaction between zein and CsA through hydrogen bonding among the amino acid groups present on both CsA and zein chains. An endothermic peak around 85° C. and glass transition temperature (T_{g}) around 175° C. was observed in the differential scanning calorimetry (DSC) thermogram of zein NP which is shown in FIG. 3. However, no change in the thermogram (endothermic peak and T_{g}) was observed with zein-CsA NP, possibly suggesting that the incorporation of CsA has not induced any structural changes in the zein backbone, and has not affected their chain flexibility or mobility.

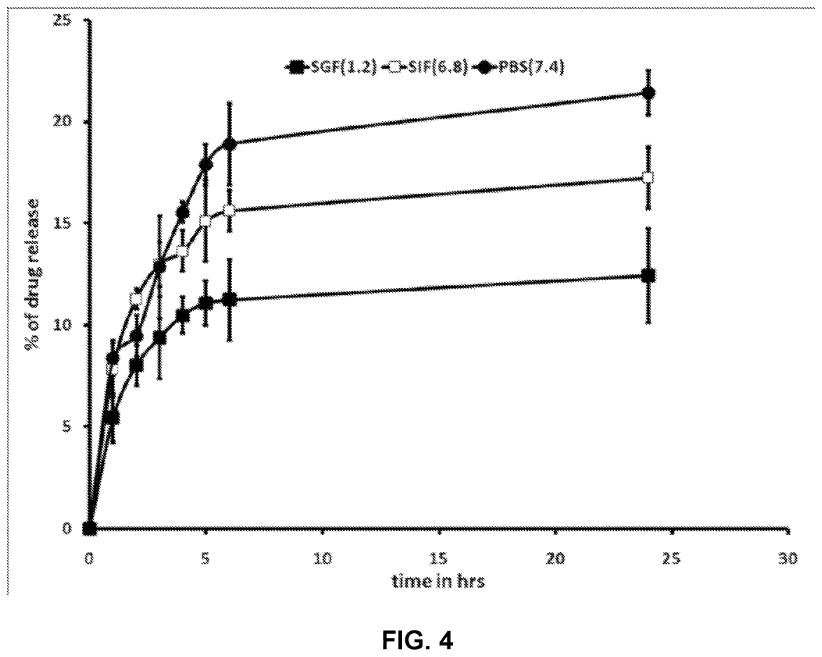

A pH-dependent in-vitro drug release profile of cyclosporine A loaded zein nanoparticles in simulated gastric fluid (SGF, pH 1.5), simulated intestinal fluid (SIF, pH6.5) and phosphate buffered saline (PBS, pH 7.4), by dialysis method, FIG. 4. One milligram of freeze dried NP were dispersed in 45 mL of SGF/SIF/PBS at 25° C. and 100 rpm in separate experiments. An aliquot (100 μL) of the supernatant was periodically removed, CsA concentration in the samples was determined by HPLC and cumulative amount released vs time was plotted. The experiment was conducted in triplicate (n=3) and data were analyzed by one-way ANOVA where p<0.05 was considered significant.

Drug Loading and CsA Release from Zein NP

CsA was loaded into the zein NP at varying protein:drug ratios (w/w) and the resultant NP were characterized in terms of particle size, zeta potential and stability. As the amount of CsA was increased, the mean particle size of zein NP increased and the stability of the particles was compromised. Z4C2 formulation with 1:0.2 (w/w) protein/drug ratio was found optimal in terms of stability. These particles displayed a Z average of 197 nm in comparison with 165 nm obtained with drug free zein NPs (Z4) as shown in Table 2.

TABLE 2

| Sample | | | Encapsulation | Loading capacity |
|---|---|---|---|---|
| code | Zein (mg) | CsA (mg) | Efficiency (EE %) | (LC %) |
| Z4 | 25 | 0 | — | — |
| Z4C1 | 25 | 2.5 | 45 ± 2.5 | 3.2 ± 0.5 |
| Z4C2 | 25 | 5 | 54 ± 2 | 10 ± 0.9 |
| Z4C3 | 25 | 7.5 | 57 ± 3.2 | 17 ± 1.2 |
| Z4C4 | 25 | 10 | 69 ± 1.5 | 27 ± 1.5 |

As the protein:drug ratio varied, EE also changed drastically. Even though Z4C4 formulation showed maximum EE of (~70%), shelf-stability of this formulation was poor. Z4C4 formulation precipitated within 2 days after the preparation, whereas Z4C2 formulation with around 55% EE displayed better stability profile and was therefore chosen for further investigations.

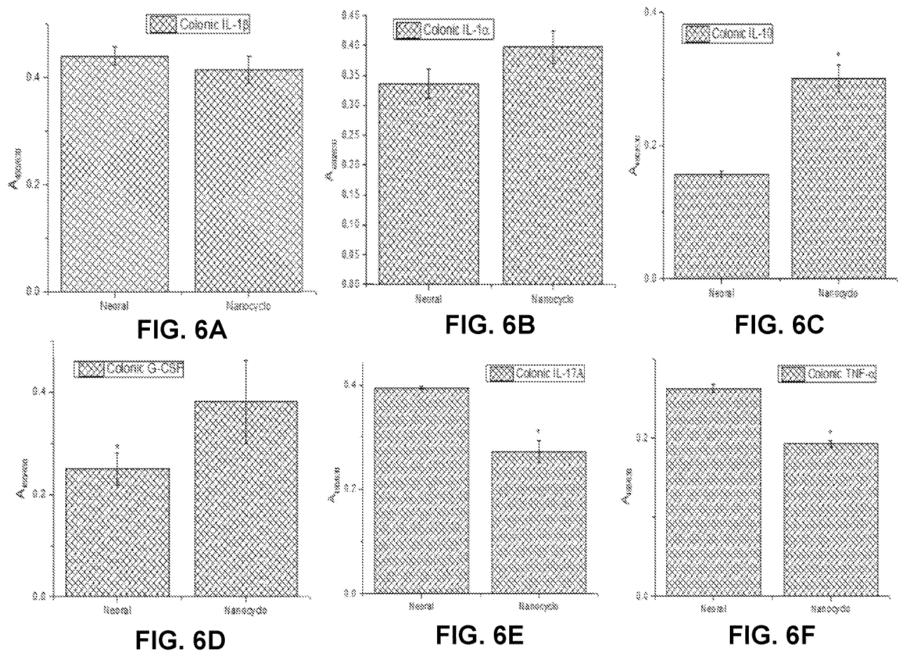

In vitro release of CsA from zein NP (Z4C2) was performed at pH 1.2, 6.8 and 7.4 to stimulate drug release in gastric, intestinal and physiological pH respectively as shown in FIG. 6. Rate of CsA release from the NP was pH dependent, with highest release in PBS pH 7.4 followed by 6.8 (SIF) and least at pH 1.2. These results may be accounted for the effect of proteolytic enzyme (pepsin and pancreatin) on da of zein NP and the resultant drug release, and the isoelectric point (pI) of zein is 6.2, and is soluble in aqueous solution only above pH 11.5. On the other hand, solubility of CsA does not change with pH since it lacks ionizable functional groups.

Although zein and CsA are hydrophobic, the proteolytic enzymes (pepsin in SGF; and pancreatin in SIF) act on the zein NP and result in initial rapid release in the first 6 h. However, the cumulative percent release was only about 11% and 15% in 24 h in SGF and SIF, respectively, presumably due to electrostatic and hydrophobic interactions between zein and CsA as well as longer duration for hydration of zein NP as compared to zein by itself.

Quantification of CsA in the colon vs serum: Concentration of CsA in the GIT (intestine, colon and cecum) and the serum samples collected 24 h after the last treatment from all the treatment groups was tested by HPLC method. As shown in Table 3, CsA levels in the colon were 3-7 fold higher in the mice treated with zein NP than the marketed formulation, NEORAL.

TABLE 3

| | Colon. conc. | Blood conc | |
|---|---|---|---|
| Formulation | (μg/g) | (μg/mL) | Colon: Blood ratio |
| NEORAL | 17.83 ± 10.34 | 5.87 ± 1.76 | 3.48 ± 2.54 |
| NANOCYCLO | 123.8 ± 36.69 | 3.8 ± 0.70 | 32.3 ± 8.6 |

Similarly, colon:serum ratio of drug concentration was also significantly high with zein NP as compared to the reference standard, NEORAL (p<0.01).

### Assessment of Disease Activity Index (DAI):

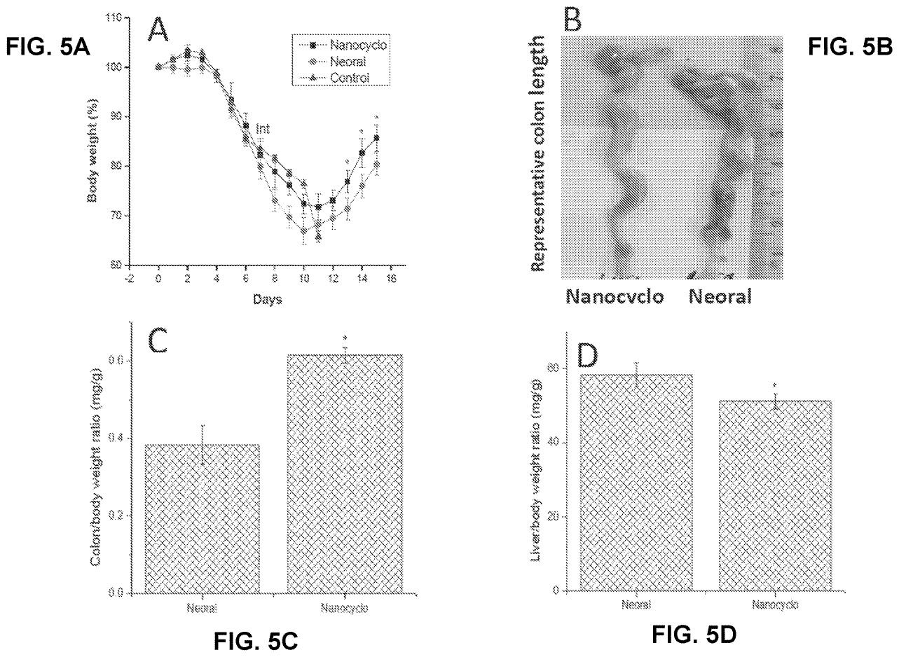

Disease activity was assessed based on weight loss, presence of diarrhea with blood or mucus, and shortening of the colon. Percent weight loss was calculated considering the body weight on day 0 as 100%. If the weight loss is more than 25% of body weight, the mice were euthanized per the protocol approved by Texas A&M University Institutional Animal Use and care Committee (IACUC) guidelines. As shown in FIG. 6, Administration of 3% DSS in drinking water slowly decreased the body weight of mice in all the test groups. Intervention with test formulations started on day 7 and continued for one week. It should be noted that body weight of the mice continually decreased till day 10, and started improving thereafter in NEORAL and NANOCYCLO groups. Because of anorexia, excessive loss of body weight (>20%) and bleeding, mice in the control group were euthanized on Day 11. Mice treated with NANOCYCLO formulation have shown remarkable recovery with a statistically significant difference in body weight as compared to the marketed formulation, NEORAL (FIG. 5A, p<0.05).

As inflammation leads to tissue destruction, reduction in colonic length is one of the accepted indicators of the extent of inflammation during DSS-induced colitis. Therefore, we determined the length and weight of the colon in the treated groups of mice. The average colon length of NEORAL colitis group of mice were to 6±1 cm compared to 8±1 cm in nanocyclo group of mice (FIG. 5B) indicating approximately 30% increase in colon lengths in mice treated with NANOCYCLO formulations (p<0.10). There were further corroborated by the data shown in FIG. 5C where colon/body weight ratios were significantly increased in mice treated with NANOCYCLO formulation (0.62±0.02 mg/g) as compared to Neoral (0.38±0.05 mg/g). As colitis led, systemic inflammation leads to hepatitis with significant increase in infiltrating immune cells. This leads to increase in liver weight, which is another indicator of inflammation status during colitis. Therefore, we determined the liver/body weight ratio in the treated group of mice. Our data indicate that liver/body weight ratio was higher in control colitis mice than in healthy mice. Moreover, liver/body weight ratio in colitis mice treated with NEORAL and NANOCYCLO treatment were 58±3 mg/g and 51±2 mg/g, respectively as shown in FIG. 5D. These indicate that treatment of colitis mice with NANOCYCLO or NEORAL formulation significantly decreased the liver/body weight ratio as compared to control colitis mice where the decrease was more in NANOCYLO group compared to NEORAL (p<0.01).

### Pro- and Anti-Inflammatory Cytokines in the Colon:

To determine the effects of NANOCYCLO or NEORAL formulation in DSS-induced colitis in mice, we determined the expression of pro-inflammatory cytokines (TNF-α, IL-1β, IL-1α, IL-17A) and anti-inflammatory cytokines (IL-10, G-CSF) in colon following treatment with NANOCYCLO and NEORAL to different groups of colitis mice and the results are presented in FIG. 6. Produced primarily by the cells of innate immune system during acute inflammation, both IL-1α and IL-1β act as acute inflammation markers. Our data showed a non-significant increase in IL-1α and a non-significant decrease in IL-1β among NANOCYCLO colitis group compared to NEORAL group (FIGS. 6A&B). TNF-α is a typical pro-inflammatory cytokine produced primarily by macrophages and other antigen presenting cells that drives the inflammation during ulcerative colitis. Our data showed a significant decrease in TNF-α in NANOCYCLO colitis group compared to NEORAL group. The marker of chronic inflammation IL-17A implicated in IBD were also significantly decrease in NANOCYCLO colitis group compared to NEORAL group. The colonic anti-inflammatory cytokines IL-10 and G-CSF on the other hand were significantly downregulated NANOCYCLO colitis group compared to NEORAL group. Together, FIG. 6 indicates a significant decrease in the markers of chronic inflammation and a significant increase in the markers of anti-inflammation in Nanocyclo colitis group thereby indicating that the nano formulation had anti-inflammatory impact of colonic mucosa during colitis recovery phase (P<0.05).

### DISCUSSION

Intravenous corticosteroids use is the standard care for severe UC flare-ups, but remission is seen only in about 40% of the patients, and the rest of the patients have to be given either CsA or undergo colectomy. While the currently marketed CsA formulations are effective, systemic absorption of the drug leads to serious side effects and their use is generally restricted for short-term treatment of about 3 months. It is therefore necessary to develop colon-specific formulations with sustained CsA delivery to the inflamed mucosa which would allow targeted effect and minimize systemic absorption, consequently avoiding the side effects. Towards this end, we developed CsA-loaded zein NP, with improved in vitro stability, drugs release properties and tested its efficacy in DSS-induced colitis model.

Preferential accumulation of nanoparticles in the inflamed regions in UC because of increased mucus production, disrupted intestinal barrier and uptake by the immune cells infiltrated into the inflamed sites has been well documented. Most of the NP formulations tested in experimental models of UC were derived from polymers such as chitosan, PLGA and methacrylate co-polymers such as Eudragits. Chitosan is a naturally occurring polysaccharide with mucoadhesive properties and chitosan surface confers a net positive charge through an electrostatic affinity to the negatively charged mucosal surfaces. However, studies have indicated that uncharged particulate surfaces demonstrate superior translocation and deposition in inflamed mucosal tissues and have relatively low translocation into healthy tissues, indicating that chitosan NP is not a favorable platform for IBD treatment. PLGA is a biodegradable polymer and is an interesting choice for encapsulating lipophilic compounds. Immunosuppressive drugs (CsA and tacromulius) encapsulated PLGA NPs were evaluated on several experimental colitis models. With neutral surface properties, PLGA could be an interesting choice for targeting inflamed intestinal regions. However, the use of stabilizers such polyvinyl alcohol (PVA), may be disadvantageous and the removal of residual PVA from the PLGA NPs can be a tricky process. Finally, the cost of synthetic polymers such PLGA is a matter of concern for developing commercially viable oral formulations for chronic conditions. NP derived from zein can fit well into this arena and could serve as much better platform for encapsulation of lipophilic compounds than chitosan or even PLGA. Based on its mucoadhesive, biodegradable properties added with low cost and ease for manufacturing protocols, zein could serve as an excellent platform for the encapsulation and stabilization of CsA.

A stable lyophilized zein NP formulation was prepared using mannitol as cryoprotectant. In addition, the pH responsive release of CsA was observed with maximum drug release at pH 7.4. Large intestinal pH ranges from (pH 7.0-7.4) and drug release at this pH could be very effective in treating IBD. A simple liquid-liquid dispersion method for the efficient preparation of CsA-loaded zein NP was developed. Formulation parameters were developed that imparted stability and re-dispersbility to the zein-CsA NP. Drug release studies in SGF, SIF and PBS pH 7.4, demonstrated pH-sensitive response with minimal release under stomach condition (SGF), and gradual increase in cumulative percent drug release with increase in intestine condition (SIF) and ileum environment (pH 7.4), showing slow and sustained release thereafter.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed, for example a range of the number of carbon atoms, molar ratios, temperatures, and the like, it is intended to disclose or claim individually each possible number that such a range could reasonably encompass, including any sub-ranges encompassed therein. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure, for example, to account for a reference that Applicants are unaware of at the time of the filing of the application.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

1. A method of preparing a nanoparticle-active pharmaceutical ingredient adduct, the method comprising:
contacting a nanoparticle with a solvent under conditions suitable for the formation of a solvent-nanoparticle mixture;
contacting the nanoparticle within the solvent-nanoparticle mixture with an active pharmaceutical ingredient under conditions suitable for forming a nanoparticle-active pharmaceutical ingredient adduct;
contacting the nanoparticle-active pharmaceutical ingredient adduct with an adduct stabilizing agent to form a stabilized nanoparticle-active pharmaceutical ingredient adduct; and
recovering the stabilized nanoparticle-active pharmaceutical ingredient adduct.

2. The method of claim 1, wherein the active pharmaceutical ingredient comprises antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, receptor blockers and reuptake modulators, beta-adrenergic blockers, calcium channel blockers, muscle relaxants, analgesics, antipyretics, stimulants, anticholinesterase agents, parasympathomimetic agents, hormones, anticoagulants, antithrombotics, thrombolytics, immunosuppressants, hormone agonists/antagonists, vitamins, antineoplastics, cathartics, diuretics, fungicides, ectoparasiticides, antiparasitics, alkaloids, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, anti-inflammatories, wound care products, antithrombogenic agents, antiangiogenic agents, antigenic agents, rejection/anti-rejection drugs, antifungal agents, antiviral agents, antibiotics, cholesterol-reducing drugs, antitussives, histamine-blocking drugs, monoamine oxidase inhibitors, or combinations thereof

3. The method of claim 1, wherein the active pharmaceutical ingredient comprises an antibiotic.

4. The method of claim 1, wherein the nanoparticle has a particle size ranging from about 120 nm to about 800 nm.

5. The method of claim 1, wherein the nanoparticle has a polydispersity index of from about 0.005 to about 0.4.

6. The method of claim 1, wherein the nanoparticle has a zeta potential of from about −15 mv to about +30 mv.

7. The method of claim 1, wherein contacting occurs with continuous agitation.

8. The method of claim 1, wherein recovering the stabilized nanoparticle-active pharmaceutical ingredient adduct comprises a solid-liquid separation method.

9. The method of claim 1, wherein the adduct stabilizing agent comprises polymers, surface active agents or combinations thereof.

10. The method of claim 1, wherein the adduct stabilizing agent comprises sodium lauryl sulfate, polyvinyl pyrrolidone, PLURONICS F68, PLURONICS F127, TWEEN 80, hydroxypropyl methylcellulose, polyvinyl alcohol, polyethylene glycol, cyclodextrin, tocopherol, succinate, hydroxyethyl cellulose, methylcellulose, carboxymethylcellulose sodium, sodium alginate or combinations thereof.

11. The method of claim 1, further comprising lyophilizing the stabilized nanoparticle-active pharmaceutical ingredient adduct in the presence of a cryopreservation agent.

12. The method of claim 11, wherein the cryopreservation agent comprises dimethyl sulfoxide, mannitol, ethylene glycol, glycerol, 2-methyl-2,4-pentanediol, propylene glycol, sucrose, trehalose or combinations thereof.

13. A method of preparing an immunosuppressive agent delivery system comprising:
contacting a zein nanoparticle with an alcohol under conditions suitable for the formation of a solvent zein nanoparticle mixture;
contacting the zein nanoparticle within the solvent zein nanoparticle mixture with an immunosuppressive agent under conditions suitable for forming a zein nanoparticle immunosuppressive agent adduct wherein the immunosuppressive agent comprises cyclosporine A;
contacting the zein nanoparticle immunosuppressive agent adduct with an adduct stabilizing agent to form a stabilized zein nanoparticle immunosuppressive agent adduct, and
recovering the stabilized zein nanoparticle immunosuppressive agent adduct.

14. The method of claim 13, wherein the solvent comprises methanol, ethanol or combinations thereof.

15. The method of claim 13, wherein the polydispersity index of the zein nanoparticle ranges from about 0.005 to about 0.212.

16. The method of claim 13, wherein the adduct stabilizing agent comprises a mixture of polyvinyl pyrrolidone and cyclodextrin.

17. The method of claim 13, further comprising lyophilizing the stabilized zein nanoparticle immunosuppressive agent adduct in the presence of a cryopreservation agent.

18. The method of claim 17, wherein the cryopreservation agent comprises mannitol.

19. The method of claim 13, wherein the ratio of zein nanoparticle to cyclosporine A is about 1:0.3.

20. A method of treating a subject comprising administering a formulation, the method comprising the stabilized zein nanoparticle immunosuppressive agent adduct of claim 13.
